(19) United States
(12) Patent Application Publication
(10) Pub. No.: US 2002/0193777 A1
(43) Pub. Date: Dec. 19, 2002

(54) Device for connection between a vessel and a container and ready-to-use assembly comprising same

(76) Inventor: Antoine Aneas, Menetrol (FR)

Correspondence Address: Monique A Morneault, Wallenstein & Wagner, 53rd Floor, 331 South Wacker Drive, Chicago, IL, 60606, US

(21) Appl. No.: 10/149,397

(22) PCT Filed: Oct. 16, 2001
(86) PCT No.: PCT/FR01/03198

(30) Foreign Application Priority Data
Oct. 17, 2000 (FR) 00/13297

(51) Int. Cl. A61B 19/00

(52) U.S. Cl. 604/411

(57) ABSTRACT

This device comprises a base (10) adapted to be mounted on the recipient (2) and comprising a sleeve (11) forming an internal bore (A), and a piston (20) adapted to slide in the bore (A), between a first position disengaged with respect to a stopper (5) of the recipient (2) and a so-called transfer position in which a hollow needle (22) borne by or constituting the piston (20), traverses the stopper (5). The piston is equipped with a valve (V) for controlling the flow of a fluid from or towards the internal volume of the recipient (2), this valve (V) being integrated in a part (26d) of the piston provided for connection of the container (3) to the needle (22).

The invention relates to a device for connection between a closed recipient and a container. It also relates to a ready-to-use assembly comprising, inter alia, a closed recipient and a connection device of the afore-mentioned type.

In the field of drug-packaging, it is known to store a component of a pharmaceutical preparation, such as for example its active ingredient, in a recipient closed by a stopper made of relatively non-rigid material, for example elastomer. A liquid may be introduced in this recipient after perforation of the stopper in order to dissolve the component contained in the recipient or place it in suspension, so as to obtain a preparation, in particular a medicament or a vaccine, in liquid form and ready to be administered to the patient.

WO-A-97/10156 discloses a connection device comprising a base adapted to cover the neck of a recipient and extending in a flange or bush forming an inner bore while a piston is mounted to slide in this bore. The piston bears a needle provided to traverse the stopper of the recipient in a so-called transfer position. This device is satisfactory and, in particular, allows a syringe provided with a luer-type connector to be connected.

However, certain products used, for example, in chemotherapies for treating cancer and incorporating molecules of cytotoxic type, are packed in recipients not necessarily corresponding to a dose having to be administered to a patient at one time. The nursing staff must therefore proceed with a multi-dosage of the product in question, i.e. an administration several times. Due to their toxic natures, these products must be handled with the greatest care and under conditions avoiding exposure of the nursing staff thereto.

It might be envisaged to mount on the known device of WO-A-97/10156 a non-return valve preventing the flow of the liquid inside the recipient towards the outside. However, such an assembly would lead to additional manipulations by the nursing staff, when the medicament is being prepared, in order to fix the valve on the device, with risks of mishandling. In addition, a risk of untimely disconnection of such a valve with respect to the device would remain, which might prove dangerous in the case of the recipient being disposed above a syringe when the piston of this syringe is pulled in order to suck the reconstituted liquid present in the recipient. In effect, in that case, a possible leakage at the level of the interface between the piston and the valve would lead to a flow of toxic product on the nursing staff's hands. Finally, the space requirement of such a valve mounted on the piston of the known device would prevent the cap of this device from being used for protecting the piston and the inner bore of the base between two samplings of products, which would increase the risks of contamination, in particular in a hospital environment.

It is an object of the present invention to solve these problems by proposing a novel connection device which allows the internal volume of the recipient to be efficiently isolated with respect to the ambient atmosphere after the piston has been brought into a position of perforation of the stopper of the recipient, without using an added valve likely to generate leakages and/or hinder manipulations of the device.

To that end, the invention relates to a device of the afore-mentioned type, of which the piston is equipped with a valve for controlling the flow of a fluid from or towards the internal volume of the recipient, this valve being integrated in a part of the piston provided for connection of a container with the needle of this piston.

Thanks to the invention, the valve, which forms a non-return valve, is permanently in place on the piston and there is no risk of untimely disconnection, hence a limitation of the risks of leakage. Due to its integration in that part of the piston provided for connection of the container, this valve does not substantially modify the dimensions of the piston, which is advantageous as far as the overall space requirement of the device is concerned.

According to advantageous aspects of the invention, the device incorporates one or more of the following characteristics:

The valve comprises a valve body in one piece with the piston and a flap valve captive of this body. In that case, the body advantageously forms an inner shoulder adapted to receive in abutment a bearing surface or portion of the flap valve. This inner shoulder serves to limit a movement of the flap valve and to close the liquid flow channel or channels inside the valve. According to an advantageous embodiment, the shoulder may penetrate superficially in a bearing portion provided on the flap valve.

The valve comprises a flap valve elastically deformable at least in a direction parallel to the direction of displacement of the piston in the bore.

The flap valve is provided with longitudinal grooves forming, in cooperation with the inner surface of the body of the valve, channels for circulation of fluid from or towards the internal volume of the recipient. In that case, the flap valve advantageously comprises a peripheral recess separating two blocks in which longitudinal grooves are made.

The flap valve comprises two end sections of diameter greater than the nominal diameter of an intermediate section lying between the end sections.

The recess or the intermediate section may be deformed under the effect of an effort of compression undergone by the flap valve.

The flap valve is symmetrical with respect to its transverse median plane.

The valve comprises a valve body provided with means for connection of the container, this valve body being adapted to accommodate a part of the container intended to interact with a flap valve of the valve. In that case, the body is advantageously adapted to receive a male luer connector belonging to the container, this connector allowing the flap valve to be manoeuvred by exerting thereon an effort directed towards the piston.

A cap for protecting and isolating the piston from the ambient atmosphere is adapted to be mounted on the base, protecting and isolating the valve, independently of the position of the piston. Thanks to this aspect of the invention, the cap may be positioned before and after a first use of the device, in particular in order to protect it with a view to subsequent use in the case of a multi-dosage.

The invention also relates to a ready-to-use assembly comprising a closed recipient containing a product, in particular a pharmaceutical preparation, this recipient being provided with a neck whose opening is obturated by a stopper, and a connection device such as described hereinabove mounted on this recipient. Such an assembly makes it possible to conserve a component of a medicament or vaccine, particularly its active ingredient, in sterile manner, and to prepare it when required by mixing with a liquid, a multi-dosage being possible without risk of leakage.

The invention will be more readily understood and other advantages thereof will appear more clearly on reading the following description of two embodiments of a connection device in accordance with its principle, given solely by way of example and made with reference to the accompanying drawings, in which:

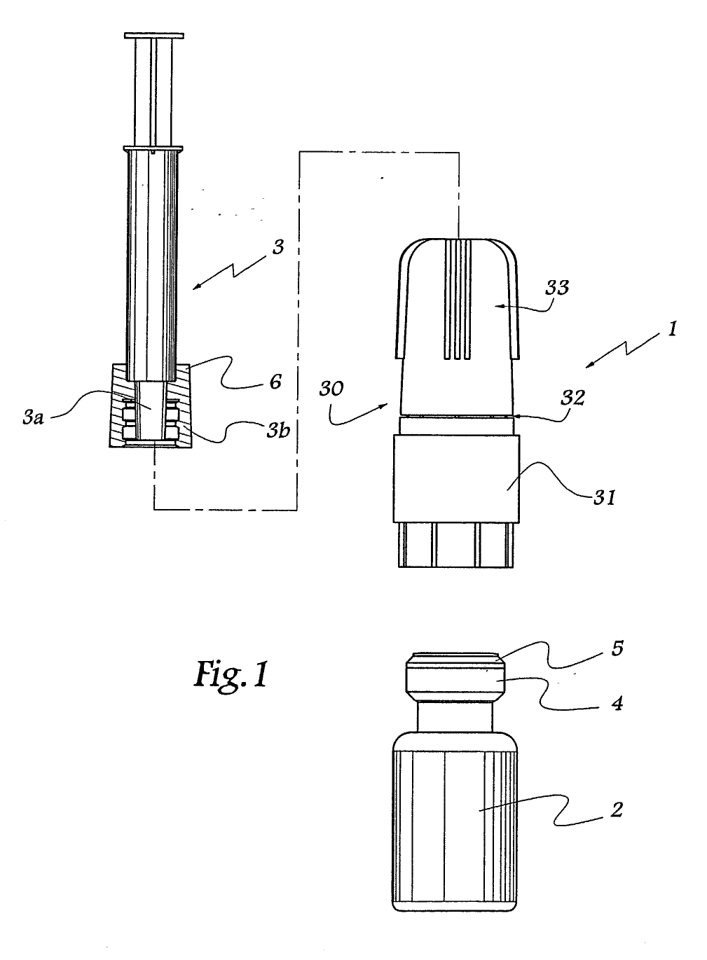

FIG. 1 is an exploded side view of a ready-to-use assembly according to the invention and of a syringe provided to cooperate with this assembly.

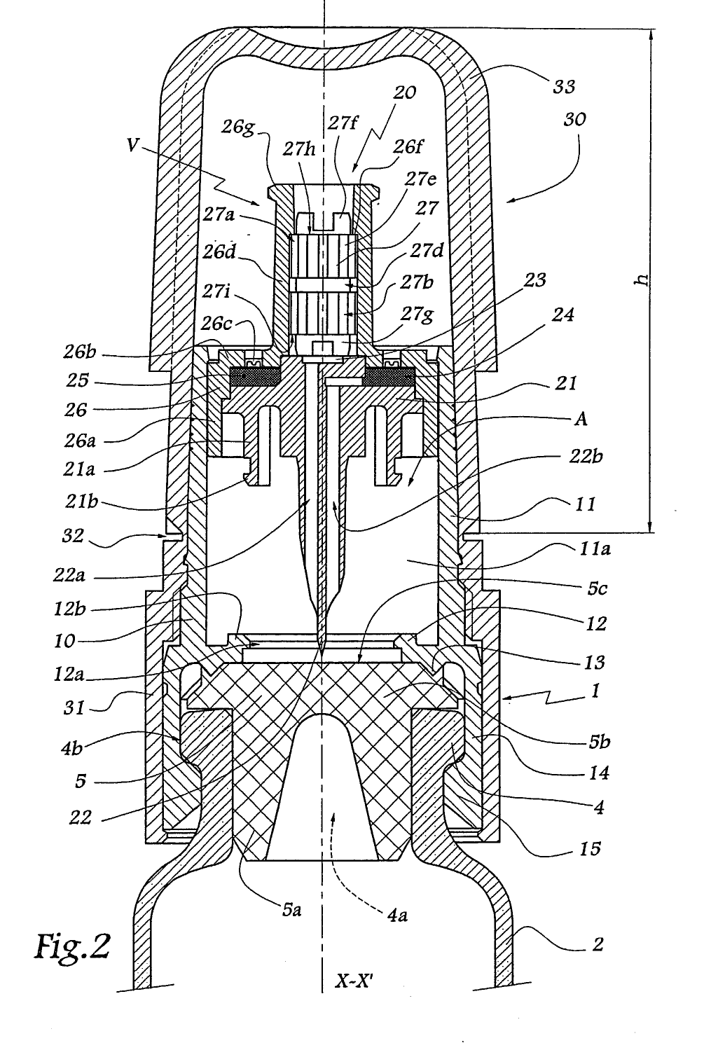

FIG. 2 is an axial section of the device mounted on the assembly of FIG. 1, in storage configuration.

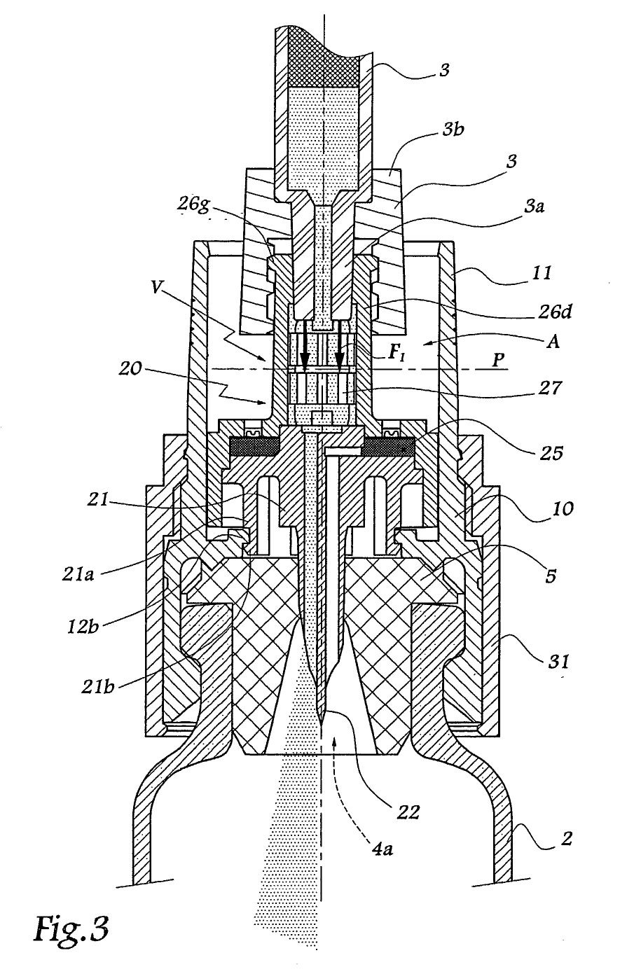

FIG. 3 is a view similar to FIG. 2 during the introduction of a liquid in the recipient by means of a syringe.

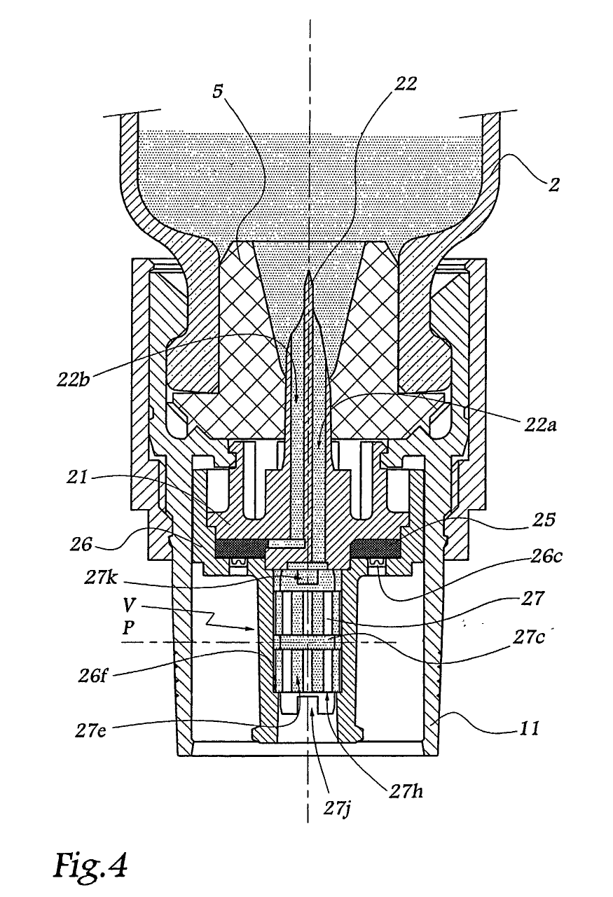

FIG. 4 is a view similar to FIG. 3 while the recipient has been upturned and the syringe has been withdrawn.

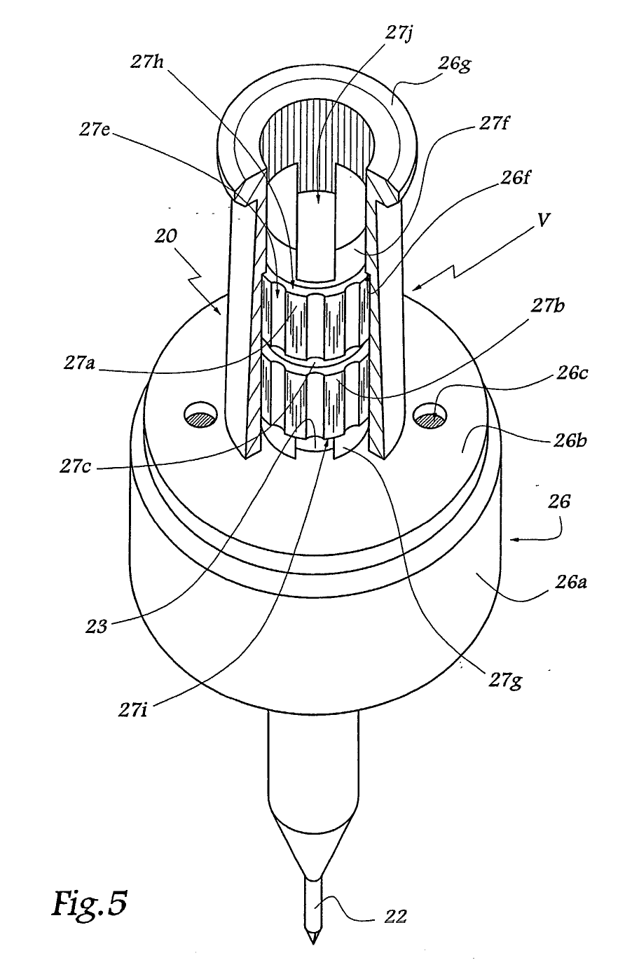

FIG. 5 is a view in perspective with parts torn away of the piston of the device of FIGS. 1 to 4.

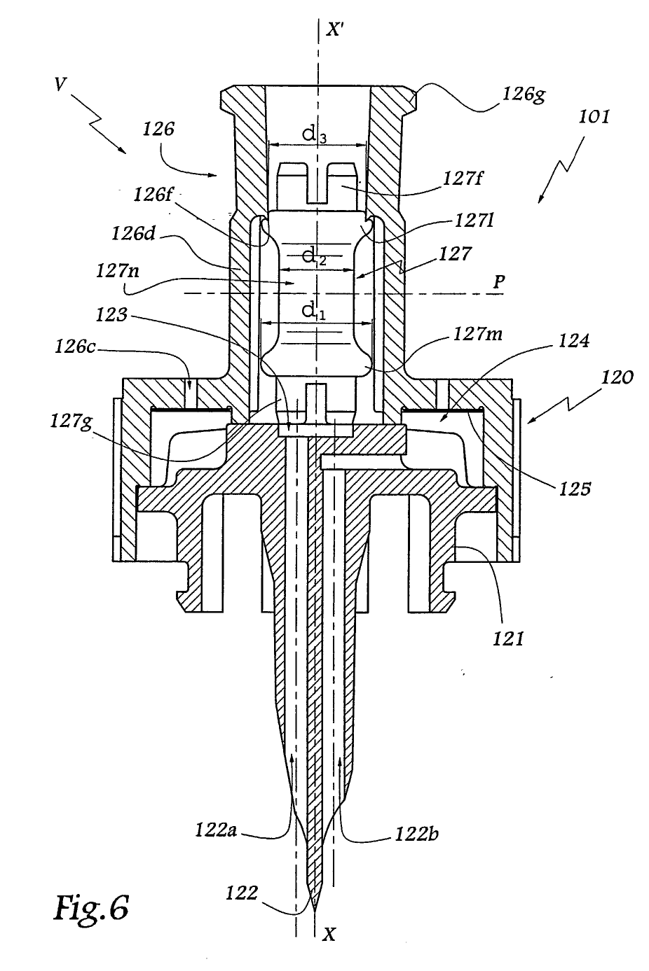

FIG. 6 is a longitudinal section of the piston of a device according to a second embodiment of the invention, with the valve integrated therein in blocked configuration.

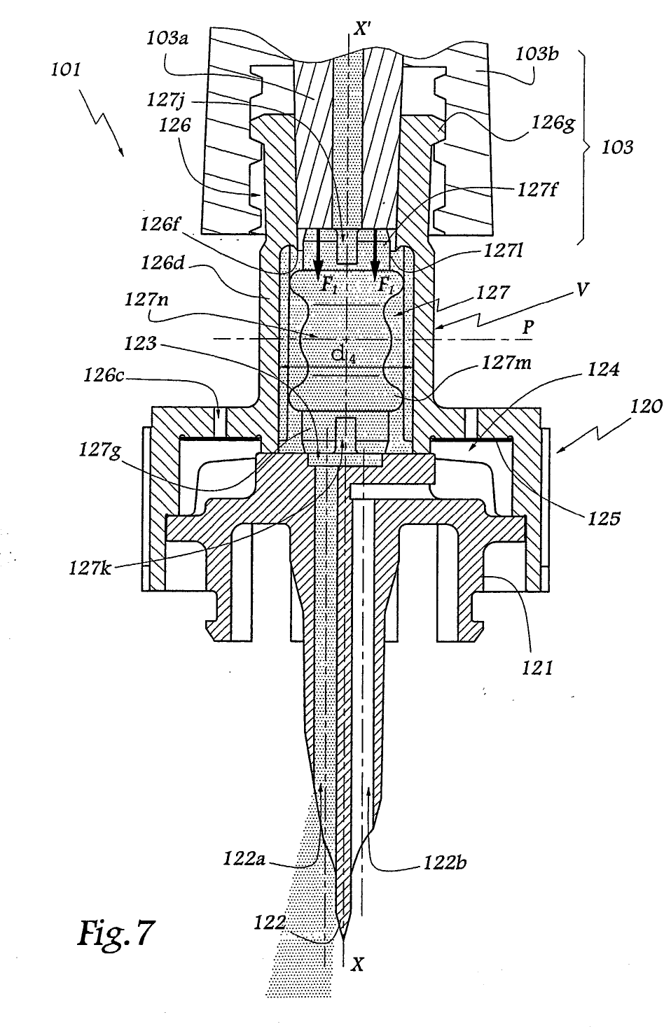

FIG. 7 is a view similar to FIG. 6 while the valve is open, and

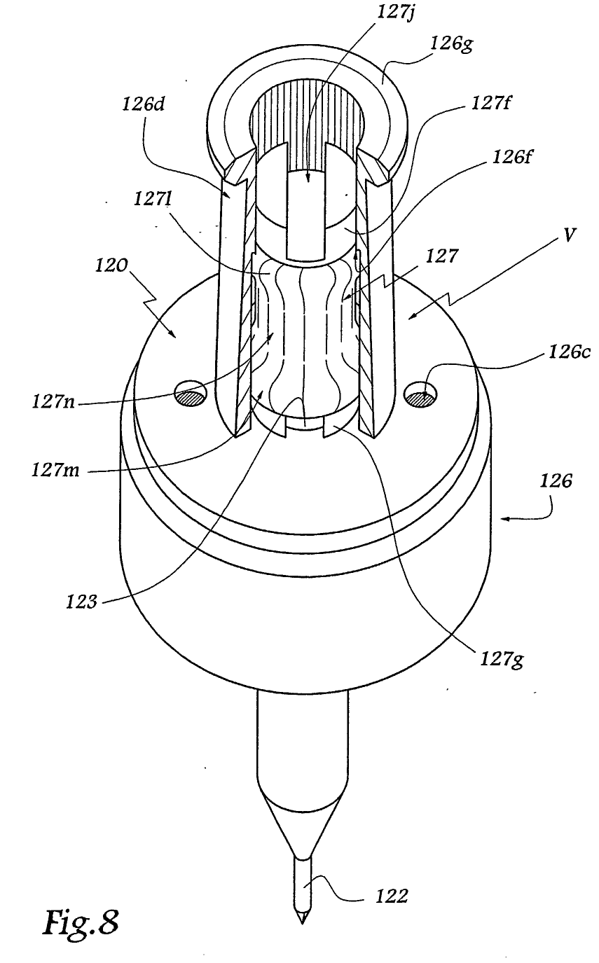

FIG. 8 is a view similar to FIG. 5 for the piston of FIGS. 6 and 7.

The device 1 according to the invention has a dual function. On the one hand, it ensures the tamper-proof nature of a recipient 2, for example a glass flask containing a product (not shown) and previously closed or stopped. This product which may be a powder contains the active ingredient of a medicament such as cytotoxic molecules used for the treatment of cancer by chemotherapy. It may be question of any other type of pharmaceutical preparation.

On the other hand, the device 1 makes it possible to ensure or establish a hermetic connection between the interior of the recipient 2 and the interior of another container such as a syringe 3 containing a liquid intended to dissolve the product contained in the recipient 2 or to place it in suspension. In place of a syringe, the container may be formed by a supple bag or another glass flask.

The flask 2 comprises a neck 4 whose opening 4a is hermetically closed by a stopper 5 made of a relatively non-rigid material, for example an elastomer, preferably rubber. According to a variant of the invention (not shown), a capsule may be arranged around the stopper 5 and an outer annular part 4b of the neck 4. The stopper 5 comprises a substantially cylindrical central part 5a adapted for a supple and hermetic fit inside the opening 4a and a flattened part 5b shouldered on part 4b and possibly covered by the capsule.

The device 1 comprises a base 10 made by injection of plastics material, for example polyethylene and/or polypropylene, intended to be mounted around the elements 4 and 5. The base 10 comprises a substantially cylindrical sleeve 11 centred on the axis of symmetry X-X′ of elements 2, 4 and 5. The sleeve 11 is connected to an annular part 12 provided with a ring 13 of pointed profile intended to penetrate superficially in the upper or exposed surface 5c of the stopper 5. Part 12 extends, opposite sleeve 11, in a plurality of elastic tabs 14 each provided with a beak 15 adapted to cooperate with the outer surface of the neck 4, with a view to elastic clipping of the base 10 on the neck.

The interior volume of the sleeve constitutes a bore A inside which may slide a piston 20 which is essentially constituted by two pieces made of moulded plastics material. The first piece 21 forms a hollow needle 22 provided with two channels 22a and 22b and adapted to perforate the stopper 5, passing through a central opening 12a in the annular part 12, when the piston 20 is displaced up to contact with the stopper 5. The channel 22a is connected to a central chamber 23 of cylindrical shape while channel 22b is connected to an annular chamber 24 in which a hydrophobic filter 25 is placed. Chamber 24 is defined between the piece 21 and a second piece 26 also forming part of the piston 20 and immobilized on piece 21 by any appropriate means, in particular by welding or adhesion.

Piece 26 comprises an annular skirt 26a which surrounds piece 21, this skirt 26a being adapted to slide against the inner surface 11a of the sleeve 11. The inner surface of the sleeve 11 and the outer surface of the skirt 26a may be shaped so that they form grooves for guiding the piston 20 in translation inside the bore A.

Piece 26 also comprises a central part 26b which defines, in cooperation with piece 21, the chamber 24 and in which are pierced vents 26c for ventilating the chamber 24.

Piece 26 also comprises, opposite skirt 26a with respect to part 26b, a sleeve 26d in one piece with the rest of the piece 26 and centred on axis X-X′.

A substantially cylindrical flap valve 27 is disposed in the inner volume of the sleeve 26b. This flap valve 27 is made of a relatively supple material such as an elastomer, for example rubber. The sleeve 27 comprises two blocks 27a and 27b separated by a part 27c of diameter smaller than that of blocks 27a and 27b. In other words, a peripheral recess 27d is provided between the blocks 27a and 27b at the level of part 27c. The recess 27d may be deformed in the case of axial compression of flap valve 27.

Flap valve 27 is symmetrical with respect to its transverse median plane P which is perpendicular to axis X-X′ and passes through the centre of the recess 27d, with the result that the flap valve may be mounted in the two directions in sleeve 26d. This facilitates assembly of the flap valve 27 in the piston 20 since no particular position has to be found before the flap valve is positioned in the sleeve.

The outer radial surfaces of the blocks 27a and 27b are provided with longitudinal grooves 27e which are substantially parallel to axis X-X′. The flap valve 27 is also provided, at its ends, with two heads 27f and 27g, of which head 27g rests against the piece 21 at the level of the chamber 23. The heads 27f and 27g present diameters smaller than those of the blocks 27a and 27b, with the result that shoulders 27h and 27i are created between these blocks and these heads.

The inner surface of the sleeve 26e also presents a shoulder 26f against which the shoulder 27h of the flap valve 27 may abut.

The dimensions of the sleeve 26d and of flap valve 27 are chosen so that, in the absence of outside stresses, the shoulder 27h of the valve 27 is in abutment against the shoulder 26f of the sleeve 26d, as shown in FIGS. 2, 4 and 5. Under these conditions, the grooves 27e of the block 27 are obturated by the shoulder 26f and the valve V formed by elements 26f and 27 is not open.

An outer envelope 30 is provided to be mounted around the base 10 and the piston 20 on the recipient 2. The envelope 30 comprises a ring 31 provided to immobilize the tabs 14 in position around the neck 4, in accordance with the teaching of WO-A-97/10156.

The ring 31 is connected by a breakable section 32 to a cap 33 which isolates the base 10 and the piston 20 from the ambient atmosphere as long as the breakable section has not been broken. It will be noted that the height h of the cap 33 is sufficient for it also to cover the valve V without interfering therewith.

Operation is as follows:

When a liquid is to be introduced inside the recipient 2, the cap 33 is withdrawn by breaking the section 32 and the piston 20 is displaced towards its position of transfer of FIG. 3, where the needle 22 traverses the stopper 5. Then, the luer connector 3a of the syringe 3 is introduced in the sleeve 26d of the valve V, while a nut 3b is screwed on an outer flange 26g of the sleeve 26d. The sleeve 26d constitutes a female luer connector for connector 3a. By tightening the nut 3d, the connector 3a is made to advance in the direction of the piece 21 of the piston 20, which is translated by an effort F₁ of compression on the flap valve 27, with the result that its shoulder 27h is moved away from shoulder 26f. Liquid can thus circulate through a central notch 27i of the head 27f then through the grooves 27e of the block 27a in the direction of the recess 27d where the flow is distributed between the grooves 27e of the block 27b before the flow is distributed again around the head 27g and penetrates through the central notch 27k of this head in the chamber 23. The valve V is then open. From the chamber 25, the liquid may be injected inside the recipient 2.

In the position of FIG. 3, tabs 21a of the piece 21 which are provided with beaks 21b cooperate with the edge 12b of the opening 12a in order to immobilize the piston in position of transfer, in accordance with the technical teaching of WO-A-98/13006.

When a sufficient quantity of liquid has been introduced in the recipient 2, it is possible to shake the latter in order to mix the liquid with the product originally present in the recipient 2 in order to obtain the desired solution or suspension. It is then possible to extract the mixture from the recipient 2 by suction thanks to the syringe 3.

When a sufficient quantity of product has been extracted, even if the recipient 2 has not been completely emptied, it is possible to loosen the nut 3b and to withdraw the connector 3a from the sleeve 3 in order to use the product contained in the syringe. By withdrawing the connector 3a from the sleeve 26d, the effort F₁ exerted on the flap valve 27 in the configuration of FIG. 3 is relaxed. Due to the relaxing of this effort and under the effect of its natural elasticity, the flap valve 27 resumes its configuration of FIG. 2 where its shoulder 27h comes into abutment against the shoulder 26f of the sleeve 26d, which leads to a closure of the valve V. The internal volume of the recipient 2 is thus isolated with respect to the ambient atmosphere and the recipient 2 may be upturned, as shown in FIG. 4, without risk of leakage of the potentially toxic mixture.

In this position, the liquid does not risk flowing via the channel 22b of the needle 22, insofar as the hydrophobic filter 25 only allows air to pass.

The channel 22b, the chamber 24 and the vents 26c allow the air to be evacuated without risk of creating excess pressure inside the recipient 2 during introduction of the liquid into the recipient 2. The risks of spashing, particularly when the connector 3a is withdrawn with respect to the sleeve 26d, are thus limited.

In the second embodiment shown in FIGS. 6 to 8, elements similar to those of the first embodiment bear identical references increased by 100.

The device 101 of this embodiment comprises a piston 120 formed by two rigidly and hermetically assembled pieces 121 and 126, defining therebetween an annular chamber 124 in which a hydrophobic filter 125 is disposed. The piece 121 also forms a hollow needle 122 of which a first channel 122a is connected to a central chamber 123, while its second channel 122b is connected to chamber 124.

As in the first embodiment, vents 126c are provided in the piece 126 for the chamber 124, similarly to a sleeve 126d in one piece with the rest of the piece 126.

The sleeve 126d is centred on an axis X-X′ which is also the central axis of the pieces 121 and 122 and the axis of slide of the piston 120 in a bore of a base (not shown), of the type such as base 10 of the first embodiment.

Inside the sleeve 126 there is disposed a flap valve 127 made of elastomer and provided with two heads 127f and 127g of the type such as heads 27f and 27g of the first embodiment. Between the heads 127f and 127g, the flap valve 127 presents two sections 127l and 127m of diameter d₁ greater than the diameter d₂ of an intermediate section 127m lying between sections 127l and 127m.

The sleeve 126d is provided with an inner shoulder 126f of pointed cross-section, the inner diameter d₃ of the sleeve 126d at the level of the shoulder 126f being smaller than diameter d₁, with the result that the shoulder 126f may abut and penetrate superficially in the section 127l of the flap valve 127, which ensures a hermetic bearing of the flap valve 127 against the shoulder 126f. In effect, the internal dimensions of the sleeve 126d and the dimensions of the flap valve 127 are chosen so that, in the absence of stress, the configuration of the flap valve is as shown in FIG. 6.

When a liquid is to be introduced in a recipient of the type such as recipient 2 of the first embodiment on which is mounted the device 101 incorporating the piston 120, the male luer connector 103a of a container 103 is introduced in the sleeve 126d and exerts on the valve 127 an effort F₁ of compression which moves the section 127l from the shoulder 126f, which has the effect of allowing liquid to flow through a central notch 127j of the head 127f then around the sections 127l, 127n and 127m, then around the head 127g and through a notch 127k of this head in the direction of the chamber 123, then of the channel 122a of the needle 122.

The configuration is in that case that of FIG. 7 where the compression of the flap valve 127 is obtained thanks to a creeping of the matter at the level of section 127n under the effect of the effort F₁, this localized deformation of the flap valve not hindering the flow of the liquid since the diameter d₂ of the section 127n at rest is substantially smaller than the inner diameter d₄ of the sleeve 126d at the level of flap valve 127.

As soon as the male luer connector 103a is withdrawn from the sleeve 126d, the valve resumes its conformation of FIGS. 6 and 8 and the valve V formed by elements 126d and 127 is again closed.

The pointed form of the shoulder 126f, which is a tapered circular lip, ensures a good tightness between the sleeve 126d and the flap valve 127, including after the valve V has been manoeuvred several times.

As before, the sleeve 126d is provided with a flange 126g allowing screwing of a nut 103b ensuring the progressive and constant nature of the effort F₁.

The flap valve 127 is symmetrical with respect to its transverse median plane P, which enables it to be mounted in the sleeve 126d without particular precaution, each section 127l or 127m being able to cooperate with the shoulder or lip 126f.

The invention has been shown with a container formed by a syringe 3. However it is applicable with other containers, for example a supple bag, as long as these containers are adapted to exert on the flap valve 27 an effort similar to effort F₁.

1. Device for connection between a closed recipient and a container, said closed recipient comprising a neck whose opening is obturated by a stopper, said connection device comprising:
a base adapted to be mounted on said recipient and comprising a sleeve forming an internal bore, and
a piston adapted to slide in said bore, between a first position disengaged with respect to said stopper and a so-called transfer position in which a hollow needle borne by or constituting said piston, traverses said stopper, characterized in that said piston (20; 120) is equipped with a valve (V) for controlling the flow of a fluid from or towards the internal volume of said recipient (2), said valve being integrated in a part (26; 126) of said piston provided for connection of said container (3) to said needle (22, 122).

2. Device according to claim 1, characterized in that said valve (V) comprises a valve body (26d; 126d) in one piece with said piston (20, 21, 26; 120, 121, 126) and a flap valve (27; 127) captive in said body.

3. Device according to claim 2, characterized in that said body (26d; 126d) forms an inner shoulder (26f; 126f) adapted to receive in abutment a bearing surface (27h) or part (127l) of said flap valve (27).

4. Device according to claim 3, characterized in that said shoulder (126a) is adapted to penetrate superficially in said bearing part (127l).

5. Device according to one of the preceding claims, characterized in that said valve (V) comprises a flap valve (27; 127) elastically deformable in at least one direction (X-X′) parallel to the direction of displacement of said piston (20; 120) in said bore (A).

6. Device according to one of the preceding claims, characterized in that said valve (V) comprises a flap valve (27) provided with longitudinal grooves (27e) forming, in cooperation with the inner surface of a body (26d) of said valve, channels for circulation of fluid from or towards the inner volume of said recipient (2).

7. Device according to claim 6, characterized in that said flap valve (27) comprises a peripheral recess (27d) separating two blocks (27a, 27b) in which said longitudinal grooves (27c) are made.

8. Device according to one of claims 1 to 5, characterized in that said valve comprises a flap valve comprising two end sections (127l, 127m) of diameter (d₁) greater than the nominal diameter (42) of an intermediate section (127n) lying between said end sections.

9. Device according to one of claims 7 or 8, characterized in that said recess (27d) or said intermediate section (127n) is adapted to be deformed under the effect of an effort of compression (F₁) undergone by said flap valve (27, 127).

10. Device according to one of claims 5 to 9, characterized in that said flap valve (27; 127) is symmetrical with respect to its transverse median plane (P).

11. Device according to one of the preceding claims, characterized in that said valve (V) comprises a valve body (26d; 126d) provided with means (26g; 126g) for connection of said container (3; 103), said valve body being adapted to accommodate a part (3a; 103a) of said container intended to interact (Fₐ) with a flap valve (27; 127) of said valve.

12. Device according to claim 11, characterized in that said body (26d, 126d) is adapted to receive a male luer connector (3a, 103a) belonging to said container (3; 103), said connector making it possible to manoeuvre said flap valve (27; 127) by exerting thereon an effort (F₁) directed towards said piston (20; 120).

13. Device according to one of the preceding claims, characterized in that it comprises a cap (30) for protecting and isolating said piston (20; 120) with respect to the ambient atmosphere, said cap being adapted (h) to be mounted on said base (10), protecting and isolating said valve (V), independently of the position of said piston.

14. Ready-to-use assembly comprising a closed recipient (2) containing a product, in particular a pharmaceutical preparation, said recipient being provided with a neck (4) whose opening is obturated by a stopper (5), and a connection device (1; 101) according to one of the preceding claims, mounted on said recipient (2).
